(19) United States
(12) Patent Application Publication
(10) Pub. No.: US 2008/0161686 A1
(43) Pub. Date: Jul. 3, 2008

(54) Methods and apparatus for controlling handheld medical devices

(76) Inventor: Nahi Halmann, Milwaukee, WI (US)

Correspondence Address: DEAN D. SMALL;THE SMALL PATENT LAW GROUP LLP, 611 OLIVE STREET, SUITE 1611, ST. LOUIS, MO, 63101, US

(21) Appl. No.: 11/590,697

(22) Filed: Oct. 31, 2006

(51) Int. Cl. A61B 8/00; A61B 8/14

(52) U.S. Cl. 600/437; 600/459

(57) ABSTRACT

A handheld medical imaging apparatus includes a handheld unit configured to be held in one hand of a user and having a clickwheel configured to be operated by a single finger of a first hand and a display for displaying a medical image. The apparatus further includes an imaging transducer configured to be held by the other hand of the user.

### BACKGROUND OF THE INVENTION

This invention relates generally to medical devices, and more particularly to methods and apparatus for controlling such devices via a simple-to-use user interface.

Portable and handheld ultrasound systems pose specific challenges for user interfaces, because the system must be operated while holding the ultrasound unit in one hand and the ultrasound transducer in the other hand. Also, many of users of portable and handheld ultrasound devices, such as surgeons, emergency physicians, primary care physicians and other clinicians are new to ultrasound and may need a simplified user interface to facilitate the adoption of these devices.

### BRIEF DESCRIPTION OF THE INVENTION

In one aspect, some configurations of the present invention provide a handheld medical imaging apparatus that includes a handheld unit configured to be held in one hand of a user and having a clickwheel configured to be operated by a single finger of a first hand and a display for displaying a medical image. The apparatus further includes an imaging transducer configured to be held by the other hand of the user.

In yet another aspect, some configurations of the present invention provide a method for obtaining medical images of patients using a handheld medical imaging apparatus. The medical imaging apparatus includes a handheld unit configured to be held in one hand of a user and having a clickwheel configured to be operated by a single finger of a first hand and a display for displaying a medical image. The apparatus further includes an image transducer configured to be held by the other hand of the user. The method includes holding the handheld unit in a first hand, holding the image transducer in the other hand, and operating the clickwheel with a single finger of the first hand to control the display of the medical image on the handheld unit.

It will be appreciated that some configurations of the present invention provide a simplified user interface for handheld device that allows a user to use a single finger for all or most of the main operations of the device. Thus, the other hand is free to hold a transducer and scan a patient. In addition, single finger operation does not require the user to look at a keyboard to find the required control. Because the user interface is similar to an interface used in popular MP3 audio players, it will look familiar to new users of ultrasound devices, thereby simplifying operation of the device for new users. In addition, various configurations of the present invention have low manufacturing costs and small overall size and weight.

### BRIEF DESCRIPTION OF THE DRAWINGS

FIG. 1 is a drawing of a configuration of the present invention held in the hands of a user.

FIG. 2 is a drawing of a configuration of the present invention operating in a B mode as an ultrasonic imaging device.

FIG. 3 is a drawing of the configuration of the present invention shown in FIG. 2 operating in a CFM mode of operation.

FIG. 4 is a drawing illustrating a manner in which a hand grip and stylus can optionally be provided in some configurations of the present invention.

The foregoing summary, as well as the following detailed description of certain embodiments of the present invention, will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block or random access memory, hard disk, or the like). Similarly, the programs may be stand alone programs, may be incorporated as subroutines in an operating system, may be functions in an installed software package, and the like. It should be understood that the various embodiments are not limited to the arrangements and instrumentality shown in the drawings.

### DETAILED DESCRIPTION OF THE INVENTION

As used herein, an element or step recited in the singular and proceeded with the word “a” or “an” should be understood as not excluding plural said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

This invention describes a unique type of user interface for a handheld or portable ultrasound product. The entire user interface operates the ultrasound system from a single control, such as a click-wheel, so that the system can be operated while holding it in one hand. The user may operate the click-wheel using a single finger. This is similar to the way some MP3 portable audio players operate. A single user interface element can be used for all primary imaging controls, such as Gain control, depth control, scan/freeze control, image storage control and toggling between B mode and other modes (such as Color Doppler and Spectral Doppler modes).

In some configurations of the present invention, a medical imaging device comprises an imaging transducer that can be held in one hand and a handheld unit comprising a medical imaging display and a clickwheel controller that can be held in the other hand. For an ultrasound imaging device operating in B mode, the clickwheel controller is configured to control gain or depth by movement of a finger in either direction about the rotary portion of the clickwheel controller, with a center key configured to toggle control between gain and depth. A dedicated key on the rotary portion is configured to toggle between scan and freeze when tapped, and another dedicated key stores an image when tapped. Yet another dedicated key on the rotary switches between B and CFM modes when tapped. This type of control allows for simple, single finger operation of the medical imaging device. In some configurations, the display is a touch sensitive display, and a stylus is provided to either enter a patient's name, make measurements on the image, or both.

In a CFM mode of operation, the medical imaging device in some configurations of the present invention has PRF/WF fixed in a preset, and the steer angle is fixed or set through a menu. However, position control uses the rotary. For example, the top half of the rotary is used for gain/depth control up or down position and the bottom half of the rotary is used for gain/depth control left or right position. Scan/freeze is controlled by a dedicated key on the rotary, store by another dedicated key on the rotary, and B/CFM modes yet another dedicated key on the rotary. The center button controls whether gain or depth is controlled by the rotary.

In some configurations of the present invention, the handheld unit includes a hand grip behind the clickwheel, and is weighted so that the center of gravity of the handheld unit is closer to the handgrip than the geometric center of the handheld unit. A battery pack and/or power conditioner may be stored within the hand grip to power the handheld unit, increase its weight and move the center of gravity towards the handgrip. In some configurations of the present invention, a stylus is provided and the display is touch sensitive, so that names can be entered, points, lines or angles can be selected on the image for measurement, and/or images can be labeled. The stylus can be stored in a suitable slot on the side or rear of the handheld unit, and may provide input to the handheld unit by activating a virtual on-screen keyboard and/or by handwriting recognition.

Nothing inherent in the present invention would prevent the use of a mechanically rotating clickwheel, examples of which are found in some MP3 players. However, non-mechanical clickwheels are also suitable for use in configurations of the present invention. An example of such a suitable clickwheel is one similar to clickwheels used in iPod devices by Apple Computer and described in U.S. Pat. No. 7,046,230 issued on May 16, 2006 to Zadesky et al., which is entitled “Touch Pad Handheld Device.” These clickwheels have a plastic surface comprising a “rotary,” which can operate in a polar coordinate mode, and typically also have four discrete mechanical buttons. A fifth button is also typically provided in the center of the rotary. The underside of each button is metal, so pressing the button completes a circuit on a motherboard. The touch-sensitive function of the clickwheel operates similarly to that of a laptop computer's touchpad or a capacitive sensing device. A conductive grid forming the clickwheel is separated from a user's fingers by a layer of nonconductive plastic. When the user presses on the plastic, a microcontroller detects the change in capacitance, determines where the finger is located and the speed and direction of its movement and passes that information to a microprocessor or another controller (in case the microcontroller controlling the clickwheel is not programmed to control the remainder of the handheld unit). When the finger is lifted, the microcontroller indicates that the user press has ended.

For purposes of the exemplary embodiments described below, it is assumed that a non-mechanical clickwheel is used, even though a mechanical clickwheel could be used in other embodiments.

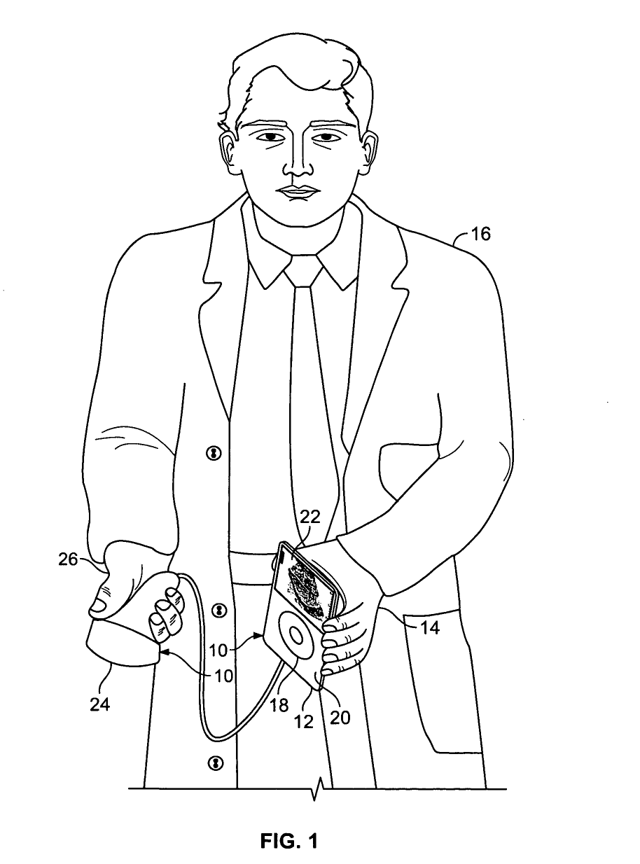

In some configurations of the present invention and referring to FIG. 1, a handheld medical imaging apparatus 10 includes a handheld unit 12 configured to be held in one hand 14 of a user 16. Handheld unit 12 has a clickwheel 18 configured to be controlled by a single finger 20 of first hand 14 and a display 22 for displaying a medical image. Apparatus 10 also includes an imaging transducer 24 configured to be held by the other hand 26 of user 16. Although more than one clickwheel 18 can be provided in some configurations, in many configurations, exactly one clickwheel 18 is provided. In configurations in which apparatus 10 is an ultrasonic imaging device, imaging transducer 24 is an ultrasonic imaging transducer.

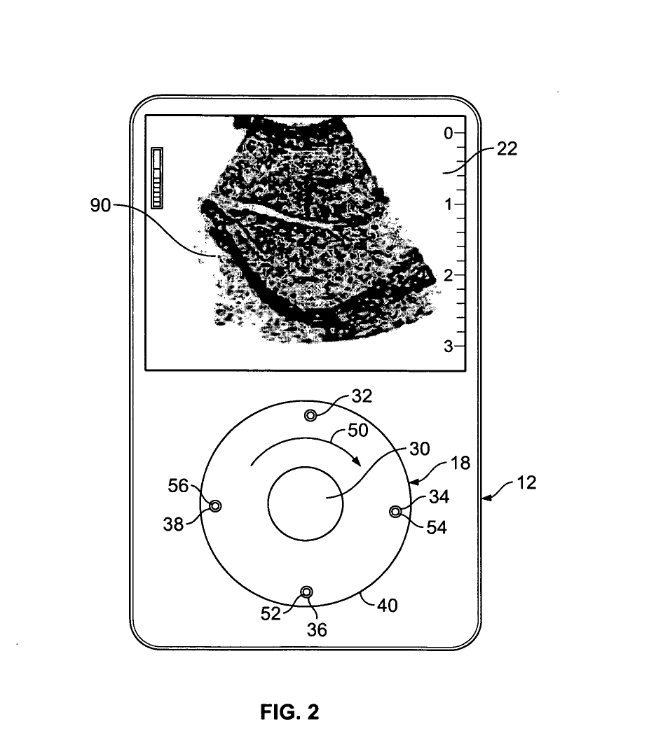

In some configurations of the present invention and referring to FIG. 2, apparatus 10 includes at least one central push key 30 and buttons 32, 34, 36, and 38 on or near clickwheel 18. In some configurations, buttons 32, 34, 36, and 38 are located around an external rim 40 of clickwheel 18. In some additional configurations, buttons 32, 34, 36 and 38 are located at the four compass directions within clickwheel 18. The latter configurations can include small breaks in a capacitive clickwheel 18 for buttons 32, 34, 36, and 38 to contact a motherboard (not shown in the drawings). Buttons 32, 34, 36, and 38 can be, for example, rubber bumps on a plastic coating covering clickwheel 18 having a metallic coating on the underside that completes a corresponding circuit on the motherboard.

In some configurations in which apparatus 10 is an ultrasound imaging system, imaging transducer 24 is an ultrasonic imaging transducer, and clickwheel 18 is configured to operate imaging controls that include gain and/or depth control 50, scan/freeze control 52, image storage control 54, and toggling between B mode and color and Doppler modes 56. Gain and/or depth control is accomplished by moving a finger clockwise or counterclockwise around clickwheel 18. Toggling between gain and depth control is accomplished by pressing central push key 30. Image storage is accomplished by pressing a button 34. Freeze and scan is toggled by pressing a button 36. Toggling between B mode and color and Doppler modes is accomplished by pressing button 38. In other configurations, the available buttons may be assigned to the same buttons differently, and/or different or additional functions may be assigned to the buttons.

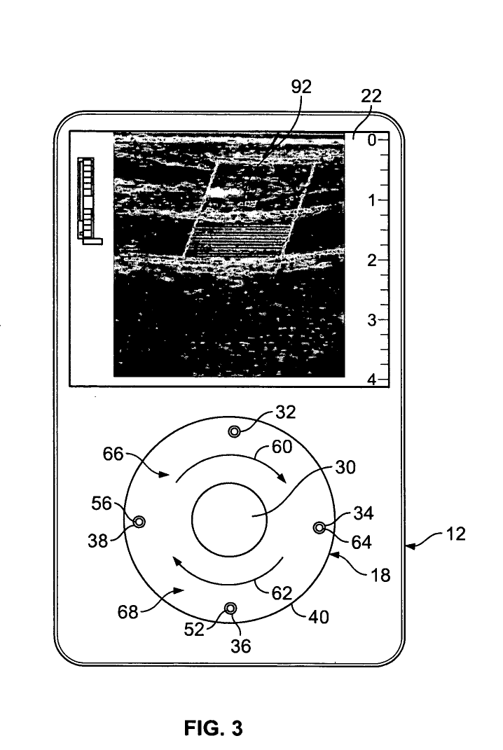

Also in some configurations of the present invention and referring to FIG. 3, clickwheel 18 is further configured to operate imaging controls that include up-down position 60 and left-right position 62, and to toggle between gain control, depth control, and position control 64, and further wherein, in a position control mode, a top portion 66 of clickwheel 18 is configured to control up/down position and a bottom portion 68 of clickwheel 18 is configured to control left-right position, or vice-versa. In other configurations, four buttons 32, 34, 36, 38 are used to control position.

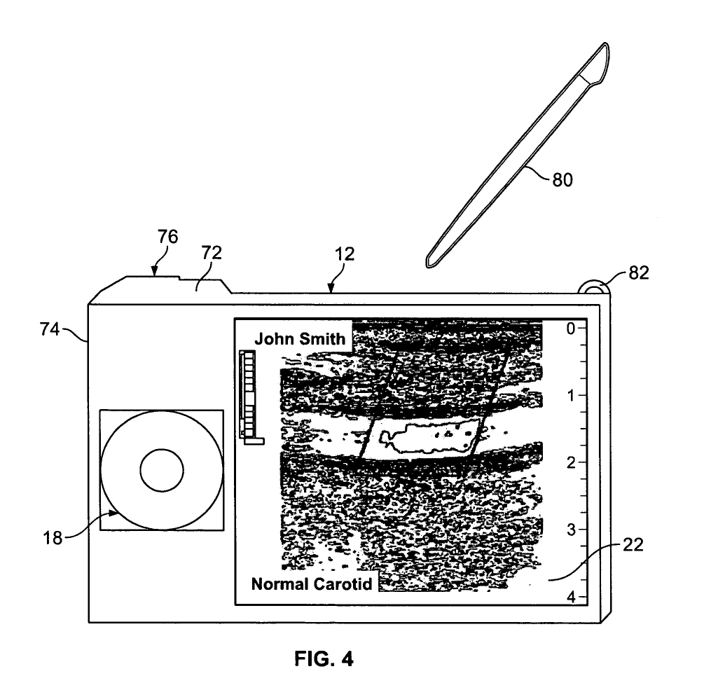

In some configurations and referring to FIG. 4, handheld unit 12 includes a housing 70 having a weighted hand grip 72 proximate one end 74 of housing 70, and click wheel 18 is located proximate hand grip end 74 of housing 70 and display 22 is located distal to hand grip end 74 of housing 70. Hand grip 72 in some configurations contains a power supply 76 for apparatus 10. Additionally, in some configurations of the present invention, handheld unit 12 can be rotated for use by both left handed and right handed users 16, by including software for orienting display 22 and interpreting click wheel 18 input appropriately for both left handed and right handed users. Furthermore, display 22 is touch-sensitive in some configurations and configured for receiving input from a stylus 80. A handwriting recognition software module can be included in apparatus 10 for use in labeling images using stylus 80.

In some configurations of the present invention, and referring to FIGS. 1, 2, 3, and 4, a method for obtaining medical images 90, 92 of patients using a handheld medical imaging apparatus 10 comprising a handheld unit 12 configured to be held in one hand 14 of a user 16 and having a clickwheel 18 configured to be operated by a single finger 20 of a first hand 14 and a display 22 for displaying a medical image 90 (or 92), and an image transducer 24 configured to be held by the other hand 26 of user 16. The method includes holding handheld unit 12 in a first hand 14, holding image transducer 24 in the other hand 26, and operating clickwheel 18 with a single finger 20 of first hand 14 to control display of the medical image on handheld unit 12. In some configurations, image transducer 24 is an ultrasonic imaging transducer. Operating clickwheel 18 can include operating a central push key 30 and buttons 32, 34, 36, and 38 around an external rim 40 of or in clickwheel 18.

In some configurations in which apparatus 10 is an ultrasound imaging system, imaging transducer 24 is an ultrasonic imaging transducer, and operating clickwheel 18 includes using the clickwheel to control gain, depth, scan/freeze, image storage and toggling between B mode and color and Doppler modes of the apparatus.

Also in some configurations of the present invention, the handheld unit 12 further includes a housing 70 having a weighted hand grip 72 proximate one end 74 of housing 70, clickwheel 18 is located proximate to the hand grip end 74 of housing 70 and display 22 is located distal to hand grip end 74 of housing 70, and holding handheld unit 12 in a first hand 14 includes holding hand grip end 74 of housing 70. In some of these configurations, hand grip 72 contains a power supply 76 for apparatus 10, and the method further includes utilizing power from power supply 76 to power apparatus 10. Also in some of these configurations, the method further includes rotating handheld unit 12 and utilizing software to orient display 22 and interpret click wheel 18 input consistent with the left handedness or right handedness of user 16.

In some configurations, entering commands to apparatus 10 comprises tapping a stylus 80 on display 22 and may further comprise utilizing a handwriting recognition software module to label images using stylus 80.

In configurations in which a stylus 80 is provided, a slot 82 may be provided in handheld unit 12 to store stylus 80 when stylus 80 is not in use.

It will thus be appreciated that various configurations of the present invention provide a simplified user interface for handheld device that allows a user to use a single finger for all or most of the main operations of the device. Thus, the other hand is free to hold a transducer and scan a patient. In addition, single finger operation does not require the user to look at a keyboard to find the required control. Because the user interface is similar to an interface used in popular MP3 audio players, it will look familiar to new users of ultrasound devices, thereby simplifying operation of the device for new users. In addition, various configurations of the present invention have low manufacturing costs and small overall size and weight.

While the invention has been described in terms of various specific embodiments, those skilled in the art will recognize that the invention can be practiced with modification within the spirit and scope of the claims.

What is claimed is:

1. A handheld medical imaging apparatus comprising a handheld unit configured to be held in one hand of a user and having a clickwheel configured to be operated by a single finger of a first hand and a display for displaying a medical image, and an imaging transducer configured to be held by the other hand of the user.

2. The apparatus of claim 1 having exactly one clickwheel.

3. The apparatus of claim 1 wherein the imaging transducer is an ultrasonic imaging transducer.

4. The apparatus of claim 1 further comprising at least one central push key and buttons around an external rim of or in the clickwheel.

5. The apparatus of claim 1 wherein the apparatus is an ultrasound imaging system, the imaging transducer is an ultrasonic imaging transducer, and the clickwheel is configured to operate imaging controls that include gain control, depth control, scan/freeze control, image storage control and toggling between B mode and color and Doppler modes.

6. The apparatus of claim 5 wherein the clickwheel is further configured to operate imaging controls that include up-down position and left-right position, and to toggle between gain control, depth control, and position control, and further wherein, in a position control mode, a top portion of the clickwheel is configured to control up/down position and a bottom portion of the clickwheel is configured to control left-right position, or vice-versa.

7. The apparatus of claim 1 wherein the handheld unit further comprises a housing having a weighted hand grip proximate one end of the housing, and the click wheel is located proximate to the hand grip end of the housing and the display is located distal to the hand grip end of the housing.

8. The apparatus of claim 7 wherein the hand grip contains a power supply for the apparatus.

9. The apparatus of claim 7 wherein the handheld unit can be rotated for use by both left handed and right handed users, and further comprising software for orienting the display and interpreting click wheel input appropriately for both left handed and right handed users.

10. The apparatus of claim 1 wherein the display is touch-sensitive and configured for receiving input from a stylus.

11. The apparatus of claim 10 further comprising a handwriting recognition module for use in labeling images using the stylus.

12. A method for obtaining medical images of patients using a handheld medical imaging apparatus comprising a handheld unit configured to be held in one hand of a user and having a clickwheel configured to be operated by a single finger of a first hand and a display for displaying a medical image, and an image transducer configured to be held by the other hand of the user, said method comprising:
holding the handheld unit in a first hand;
holding the image transducer in the other hand; and
operating the clickwheel with a single finger of the first hand to control the display of the medical image on the handheld unit.

13. The method of claim 12 wherein the image transducer is an ultrasonic imaging transducer.

14. The method of claim 12 wherein said operating the clickwheel comprises operating a central push key and buttons on sides of the clickwheel.

15. The method of claim 12 wherein the apparatus is an ultrasound imaging system, the imaging transducer is an ultrasonic imaging transducer, and said operating the clickwheel comprises using the clickwheel to control gain, depth, scan/freeze, image storage and toggling between B mode and color and Doppler modes of the apparatus.

16. The method of claim 12 wherein the handheld unit further comprises a housing having a weighted hand grip proximate one end of the housing, the click wheel is located proximate to the hand grip end of the housing and the display is located distal to the hand grip end of the housing, and said holding the handheld unit in a first hand comprises holding the hand grip end of the housing.

17. The method of claim 16 wherein the hand grip contains a power supply for the apparatus, and the method further comprises utilizing power from the power supply to power the apparatus.

18. The method of claim 16 further comprising rotating the handheld unit and utilizing software to orient the display and interpret click wheel input consistent with the left handedness or right handedness of the user.

19. The method of claim 11 further comprising entering commands to the apparatus by tapping a stylus on the display.

20. The method of claim 19 further comprising utilizing a handwriting recognition module to label images using the stylus.
